(19) United States
(12) Patent Application Publication
(10) Pub. No.: US 2013/0226062 A1
(43) Pub. Date: Aug. 29, 2013

(54) BANDAGE FOR APPLYING TO A HUMAN OR ANIMAL BODY

(71) Applicants: Michael Kloeppels, Aachen (DE); Ulrike Maass, Kaiserslautern (DE)

(72) Inventors: Michael Kloeppels, Aachen (DE); Ulrike Maass, Kaiserslautern (DE)

(73) Assignee: KARL OTTO BRAUN GMBH & CO KG, WOLFSTEIN (DE)

(21) Appl. No.: 13/880,832

(22) PCT Filed: Oct. 12, 2011
(86) PCT No.: PCT/EP11/67806

§ 371 Date: Apr. 22, 2013

(30) Foreign Application Priority Data
Oct. 21, 2010 (DE) 10 2010 042 772.1

(51) Int. Cl. A61F 13/00

(52) U.S. Cl. CPC A61F 13/00034
USPC 602/44; 602/53; 602/45

(57) ABSTRACT

A bandage for applying to a human or animal body, includes a two-dimensional tape material as base structure with a longitudinal extent, made of a textile material with a first side and a second side, wherein a cushion layer is provided on one of the two sides, at least over part of the longitudinal extent of the base structure, wherein the cushion layer is composed of threads and/or fibers which are part of the textile base structure, i.e. which are introduced into and/or drawn out of the textile base structure.

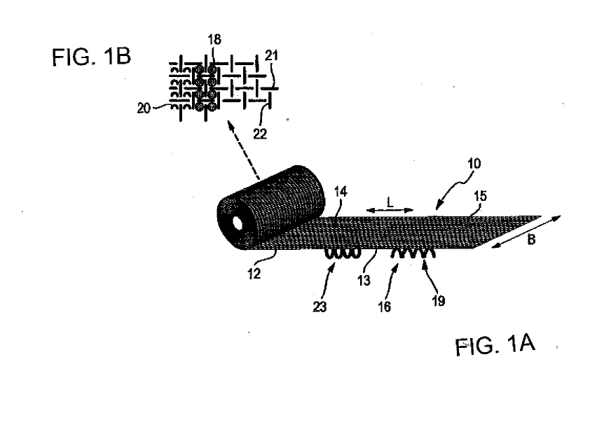

The invention relates to a bandage for applying to a human or animal body including a two-dimensional tape section as base structure with a longitudinal extent and made of a textile material with a first and a second side, wherein a cushion layer is provided on at least one of the two sides at least over part of the longitudinal extent of the base structure.

The bandage according to the invention is in particular a compression bandage or compression band.

In the state of the art, two-layered compression bandage sets are known, i.e., a set consisting of separate elements, wherein one element is a cushion bandage and the other element is a longitudinally stretchable compression bandage. The bandages are applied on top of each other. This has the disadvantage that the application process takes relatively long because two bandages have to be applied around the corresponding body part and in addition the risk that errors are made during application is significantly increased because the two bandages have to be oriented relative to one another in the desired manner. The compression is achieved by interaction between the two bandages, wherein the cushion bandage can also contribute to the compression.

In addition, one-piece compression cushion bandages are known which are made of two separate material layers, for example a laminate made of a cohesive nonwoven material and a plastic foam cushion or a nonwoven which is needled with a knitted fabric.

Thus, a compression bandage is for example known from WO 95/16416 in which a spacer layer made of threads is provided between two layers made of a knitted material.

A similar configuration of a medical bandage is known from EP 0 752 839 B1.

DE 79 29 812 U1 describes a medical cushion bandage which serves for cushioning support bandages. The cushion bandage consists of a fully synthetic nonwoven material, which consists of two layers with different fiber thicknesses.

Given the state of the art, it is an object of the invention to provide a medical bandage or band with a cushion which is easy to apply and to handle and has a good fit and in addition can be manufactured cost effectively.

The invention solves this object with a bandage or band for applying to a human or animal body including as base structure a two-dimensional tape section with a longitudinal extent and being made of a textile material with a first and a second side, wherein a cushion layer is provided on at least one of the two sides at least over a part of the longitudinal extent of the base structure, wherein the cushion layer is made of threads or fibers which are a part of the textile base structure, i.e., which are introduced into the textile base structure and/or are drawn out of the textile base structure.

The bandage or band is in particular a compression bandage or band which has an integrated cushion layer. The terms bandage and band are used synonymously in the following.

The cushion layer is formed by threads or fibers which are part of the textile base structure. In particular in a woven fabric, such threads or fibers are the weft and warp threads as well as introduced pile threads, wherein according to the invention, in particular pile fabrics can be provided. In addition, knits can also be provided as base structure, into which threads or fibers are introduced or drawn out. Finally, in the case of nonwovens i.e., not woven materials, fibers or threads can be drawn out of the textile base structure or can also be introduced for forming the cushion layer. Generally, the introduction of threads or fibers into a textile base structure or drawing threads or fibers out of a textile base structure is already known in the state of the art, for example in the carpet or automobile textile industry, but also in the clothing and home textile industry. The introduced or drawn out fibers can form a pile (in particular fiber tufts) or loops, or they can be unordered fibers.

The pile layer in this case forms the outermost layer of the bandage which layer faces a carrier of the bandage.

The term thread relates to a linear textile linear structure, wherein it is differentiated between spun yarns, produced in spinning mills and filament yarns, produced in chemical fiber companies. Threads can be made of one or multiple yarns. The term fiber includes natural staple fibers and chemical staple fibers and endless single filaments.

Such a configuration of a bandage according to the invention facilitates handling of the bandage because only a single bandage has to be applied. In addition, the fit of the bandage can be improved and pressure points can be avoided. Compared to bandages or bands without a cushion layer, bandages or bands with integrated cushion have the advantage to show less skin abrasive effects and are thus gentler. With regard to already known multilayered bandages or bands, the present configuration offers an alternative configuration which can be manufactured easily. Separately manufactured layers have the disadvantage that the two layers cannot be united and joined accurately, especially when their length and height differs individually so that the possibility of the individual configuration cannot be optimally utilized. Compared to bandages or bands which are made of two separate layers, a compression cushion bandage with integrated cushion layer has advantages regarding distribution of compression pressure because the length and width of the cushion layer can be individually adjusted, in particular in the edge region for avoiding bulges and pinch effects. A compression bandage made of multiple separate layers poses the risk that already during application, and at the latest when moving the extremity, the joined layers, for example due to delamination, detach from each other again thereby decreasing the compression effect.

Particularly preferably, the cushion layer and the textile base structure are directly adjacent one another.

The cushion layer, which is provided on at least one side of the bandage or band, is thus not made of an independent textile surface which is needled, laminated, sewed or in other ways connected in a desired manner with the base material, but the cushion layer or the cushion layers are made of threads and/or fibers, which are introduced into the base material or are drawn out of the base material. The cushion layer or cushion layers thus form the continuation of the base structure.

Beside the application of such bandages and bands in the field of medical compression bandages and bands, other fields of application such as any type of elastic or non-elastic bandages and bands are conceivable.

The cushion layer can be configured uniform and continuous over its longitudinal and/or transverse extent. The longitudinal extent of the cushion layer can differ from the longitudinal extent of the base structure. In addition, the transverse extent of the cushion layer can differ from the transverse extent of the base structure. Preferably however, the longitudinal extent and in particular the transverse extent of the base structure and the cushion layer correspond to each other.

Particularly preferably, the cushion layer is variable regarding its height, structure and/or with regard to the introduced and/or drawn out fibers and/or threads over its longitudinal extent and/or its transverse extent or is provided only in regions. The height of the cushion layer is regarded as the dimension which does not coincide with the two dimensions of the base structure. In particular regions of the cushion can be formed as pile or loops, wherein pile means that loose fiber ends are provided, whereas in the case of loops no loose fibers or fiber tufts are provided, but closed loops protrude from the base structure and form the cushion layer. The height of the pile or the loops of the cushion layer can vary over the longitudinal or transverse extension of the cushion layer continuously or section wise or pattern wise including a jacquard-like configuration of the height. In addition, variation between loops and pile configuration of the cushion layer is possible, i.e., the two can be combined. Finally, different threads or fibers can be introduced or drawn out of the base structure, in particular also different threads or fibers of different colors or different materials or different fiber or threads thicknesses, which provides additional design options.

In particular, the cushion layer can be adjusted to the respective use of the bandage or band. Thus, when using the bandage or band in compression therapy, a bandage can be constructed so that a lower pile height is provided for draping the foot or the heel of a leg, so that the bandage or band does not accumulate too much and the pile or loop height then increases in the subsequent areas. Similar considerations apply also in other applications such as for example in lymph drainages and for further human and animal body extremities.

Further, anatomy related protrusions such as bone structure of the ankle or the wrist can be treated more gently during compression therapy by corresponding recesses and adjustment of the cushion layer by adjusting the height of the cushion layer, and a higher working safety and application safety can be provided because the predetermined shape allows avoiding application errors.

Preferably, the base structure can include or in particular can be made of a woven, knitted and non woven (nonwoven). The threads and/or fibers can be introduced or drawn out by means of needling, tufting dilourizing and/or roughing up. Further, such bandages and bands can also be attained by cutting the pile fibers and/or pile threads of double layered pile textiles, so that two bandages or bands with a respective cushion layer can be obtained, or with a method similar to the weaving of Axminster carpet. In this case, fiber tufts are introduced during the Axminster weaving during the weaving process.

In the field of tufting, different configurations exist. In particular, differences in the shape of fiber tufts or loops, differences in the height of the pile or the loops and color patterns or the possibility to mix different yarn types or fiber materials.

The so called Boucle product, also referred to as tufting loop product is possible here. Further conceivable is a velour; i.e., the loops are already cut open on the tufting machine and later cleanly trimmed. This results in a flat product with even surface pattern. Beside the classical velour, soft and semi-velour are mentioned here. Velour is also referred to as cut pile product. Possible are tuft loops and tufting velour products with cross over loop and cross over cut. In all patterns, Boucle and velour, i.e. as loops but also as pile product, vertical-horizontal variants can be manufactured. Principally, hybrid forms i.e. configurations with velour (pile) and loop characteristics can be achieved, wherein nipples which have already been cut on the tufting machine and uncut nipples alternate. Further, one or further layers, as the case may be also cushion layers can in particular be arranged on the side of the base material which faces away from the cushion layer. These further layers can have cohesive or adhesive properties. As an alternative, coatings for fixing and stabilizing the base structure, but also the fibers and/or threads for the cushion layer can be provided.

In addition, using fibers or threads of different colors makes it possible for example to tuft in, weave in or introduce with the other possible methods, the logo of the customer.

Further, one or multiple layers of the base structure can be configured elastic in longitudinal and/or transverse direction. An elasticity, in particular of the warp threads of the base structure can be provided. The elasticity can be in particular in the range from 3% to 240% when measured with nondestructive force stretch test for recoding a hysteresis curve with a maximal tensile force of 10 N/cm and a force transducer of 2.5 kN, in particular in the range from 40% to 180%. The compression pressure which can be generated with the bandage/band—measured as resting pressure in vivo on the resting, lying human leg with a pressure measuring device of the company Kikuhime, between draped-on bandage and the skin at the transition from the Achilles tendon to the soleus muscle) corresponds to the measure point B1 defined in the standard RAL GZ 387) wherein two bandages are wrapped on in circular wrapping so that when the wrapping is complete, four layers overlappingly lie over the pressure sensor—is to be between 5 mmHg and 150 mmHg, preferably between 10 mmHg and 90 mmHg.

When the base structure is a woven material, it can in particular be weaved in plain weave. However, further binding types are also conceivable, in particular the binding types can also vary over the length and/or width of the base material.

Further, the bandage or band can be configured cohesive or adhesive on the side which is not provided with the cushion layer or on the cushion layer free region. This is advantageous with regard to the anti -slip properties and the improved fit and the so called self grip.

The following materials can be used for the warp thread and the heft thread of the base material, and for the threads and fibers which form the cushion layer, in particular for pile warp threads, wherein warp thread or heft thread of the base material and threads or fibers of the cushion layer can be made of the same or different materials: cotton, polyester, viscose, polyurethane, polypropylene, polyethylene, polyacrylamide (nitrile) polybutylene terephtalate polyvinyl fluoride, elastane, wool.

In the following, the invention is explained in more detail by way of a drawing.

The sole FIGURE shows a possible embodiment of a compression cushion bandage, wherein the compression cushion bandage is shown in representation B). The cushion bandage is overall designated with the reference sign 10 and has a longitudinal extent which is indicated with the double arrow L and a transverse extent which is indicated with the double arrow B. On one of the two sides 12 of a base structure 14 which is configured as two-dimensional tape section (13) and has a much greater longitudinal extent (L) than transverse extent (B), a cushion layer 16 is arranged at least over a part the longitudinal extent of the base structure 14. The further side of the base structure 14 is designated 15. The cushion layer 16 is formed by threads or fibers, which are introduced into the textile material of the base structure 14. Representation A) shows the base structure 14 which is a textile that is woven in plain weave with warp thread 21 and heft thread 22. In addition, pile threads 18 and 20 are introduced into the woven fabric, wherein these pile threads 18 and 20 have different thread thicknesses. The pile threads 18 have free fiber ends which protrude from the base structure 16 as can be seen in particular in the representation B) in the region 19. This configuration is referred to as pile or velour. In contrast, the pile threads 20 are configured in loop form, so that loops protrude from the base structure 14 (regions 23 in representation B). Together, the loops and the pile form the cushion layer 16. The cushion layer 16 is in particular attained in that the thread 18, 20 or the threads which is or are drawn out of the base structure 14 or which is or are introduced into the base structure, form an air cushion between themselves and the base structure 14 and achieve a low pressure and a certain resilience during application.

A corresponding cushion compression bandage 10, wherein the warp threads 21 are configured elastic, has the advantage of being capable of being applied easily and at the same time to be manufactured easily.

1-12. (canceled)

13. A compression bandage for applying to a human or animal body, comprising:
a base structure in the form of a two-dimensional tape material, said base structure being made of a textile material and having a first side, a second side and a longitudinal extent; and
a cushion layer provided on one of the first and second sides at least over a part of the longitudinal extent of the base structure, said cushion layer being formed by at least one member selected from the group consisting of threads and fibers, said member being part of the textile base structure and introduced into the textile base structure or drawn out of the base structure, wherein regions of the cushion layer are configured as one of a pile and loops, and wherein one or multiple plies of the base structure are elastic in a longitudinal and/or transverse direction.

14. The bandage of claim 13, wherein the cushion layer forms an outermost layer of the bandage, said outermost layer facing toward a wearer of the bandage.

15. The bandage of claim 13, wherein the cushion layer and the base structure are directly adjacent one another.

16. The bandage of claim 13, wherein the cushion layer has another longitudinal extent and is configured uniform over the other longitudinal extent on the base structure.

17. The bandage of claim 13, wherein at least one member selected from the group consisting of a height of the cushion layer, a structure of the cushion layer, the fibers introduced into the base structure and the threads introduced into the base structure, varies along a longitudinal and/or transverse extent of the cushion layer.

18. The bandage of claim 13, wherein the base structure comprises a member selected from the group consisting of a woven material, a knitted material and a nonwoven material.

19. The bandage of claim 19, wherein the base structure is formed by the member.

20. The bandage of claim 13, wherein the base structure is a pile woven fabric or a woven fabric, a knit fabric or nonwoven fabric produced with a method that introduces pile threads or pile fibers.

21. The bandage of claim 13, wherein the threads or fibers are introduced into the base structure or drawn out of the base structure by needling, tufting, dilourizing or roughening.

22. The bandage of claim 13, wherein the base structure further comprises one or more further layers.

23. The bandage of claim 22, wherein the one or more further layers are arranged on a side of the base structure which faces away from the cushion layer.

24. The bandage of claim 22, wherein the further layers are configured cohesive or adhesive.
